(19) United States
(12) Patent Application Publication
(10) Pub. No.: US 2011/0106020 A1
(43) Pub. Date: May 5, 2011

(54) Catheter For Deactivating At Least A Portion of the Digestive Enzymes In An Amount Of Bile

(76) Inventors: Lucas B. Elmer, Cincinnati, OH (US); Mark D. Overmyer, Cincinnati, OH (US); Alessandro Pastorelli, Rome (IT); Frederick E. Shelton, IV, Hillsboro, OH (US); Galen C. Robertson, Cincinnati, OH (US); Jeffrey D. Messerly, Cincinnati, OH (US)

(21) Appl. No.: 12/835,005

(22) Filed: Jul. 13, 2010

Related U.S. Application Data
(60) Provisional application No. 61/256,221, filed on Oct. 29, 2009.

(51) Int. Cl. A61M 25/00; A61B 18/04

(52) U.S. Cl. 604/284; 606/27

(57) ABSTRACT

The present invention generally provides for a catheter for surgically treating a patient. The catheter has an inlet catheter having a proximal end and an open distal end, and first and second branch sections having proximal ends attached to the open distal end of the inlet lumen. The first and second branch sections and distal ends terminating at an open proximal end of an outlet lumen. The first passageway lumen has a device for deactivating at least a portion of the digestive enzymes in an amount of bile.

### REFERENCE TO RELATED APPLICATIONS

This non-provisional application claims the benefit of U.S. Provisional Application Ser. No. 61/256,221 entitled: “Bile Manipulation System and Methods”, filed on Oct. 29, 2009.

### BACKGROUND OF THE INVENTION

The present invention relates to methods and systems for manipulation of bile within the body in order to affect a metabolic improvement.

Obesity is a disease in which excess body fat accumulates due to an imbalance between caloric intake and caloric expenditure. Obese patients often suffer from serious health issues, or co-morbidities, such as diabetes, heart disease, asthma, hypertension and many others. Many studies have shown a direct correlation between obesity and these co-morbid conditions, resulting in a finding that obesity substantially reduces life expectancy. For many obese patients, the desire to eat and presence of hunger are major factors that contribute to their caloric imbalance.

Amongst the most common co-morbidities is Type II diabetes. The most common form of diabetes, Type II, occurs in approximately 3-5% of Americans under 50 years of age, and increases to 10-15% in those over 50. More than 90% of the diabetics in the United States are Type II diabetics. Sometimes called age-onset or adult-onset diabetes, this form of diabetes occurs most often in people who are overweight and who do not exercise. It is also more common in people of Native American, Hispanic, and African-American descent. People who have migrated to Western cultures from East India, Japan, and Australian Aboriginal cultures also are more likely to develop Type II diabetes than those who remain in their original countries. Type II is considered a milder form of diabetes because of its slow onset (sometimes developing over the course of several years) and because it usually can be controlled with diet and oral medication. The consequences of uncontrolled and untreated Type II diabetes, however, are the just as serious as those for Type I. This form is also called noninsulin-dependent diabetes, a term that is somewhat misleading. Many people with Type II diabetes can control the condition with diet and oral medications, however, insulin injections are sometimes necessary if treatment with diet and oral medication is not working.

Many obese patients require some sort of intervention to overcome their obesity and to treat their accompanying co-morbidities. Behavior and lifestyle modification are often the first measures to be taken. This includes reducing caloric intake as well as increasing exercise to increase caloric expenditure. If these measures are not sufficient enough to achieve the weight loss desired by the patient, pharmaceuticals are often introduced that will suppress appetite as well as boost metabolism. Many of these pharmacological approaches result in negative side effects, which may include increased sweating, tachycardia and hypertension.

The patient's last option would be surgical intervention. Currently, one of the most common bariatric surgeries is gastric bypass with a Roux en-Y. Gastric bypass with a Roux en-Y is a procedure that is both restrictive and malabsorptive in nature. A small pouch is first created in the stomach, by way of surgical stapling, greatly restricting the amount of food that can be ingested. Next, the small intestine is transected, the distal portion is anastomosed to the stomach and the proximal portion is anastomosed back to the small intestine further downstream. This limits the amount of time that the ingested food and its nutrients can be absorbed by the body. While this procedure is very effective in creating weight loss, there are many side effects associated with it, such as nutritional deficiencies and sometimes even death.

Other approaches such as the jejunoileal bypass and biliopancreatic diversion offer excellent results in terms of rapid and long term weight loss. However, specific complications associated with these procedures include a myriad of iatrogenic nutritive deficiencies such as vitamin A deficiency, osteoporosis and protein-calorie malnutrition.

Additional drawbacks to these surgical procedures are that they are often extremely invasive such as through rerouting of larger portions of the gastrointestinal tract and these procedures require general anaesthesia thereby increasing the risk for morbidly obese patients. Furthermore, these procedures are generally considered irreversible.

What is needed therefore are means for correcting an imbalance between caloric intake and caloric expenditure in patients, as well as a means for treating co-morbidities often associated therewith, which is non-invasive or minimally invasive and which may be reversible.

### SUMMARY OF THE INVENTION

The present invention generally provides means for correcting an imbalance between caloric intake and caloric expenditure in patients, as well as a means for treating co-morbidities often associated therewith, which is non-invasive or minimally invasive and which may be reversible. More specifically, the present invention provides systems which cause metabolic improvement in a patient by controlling the amount of bile available for food breakdown or by controlling the effective absorption time and area by delivering bile to selected locations in the intestinal tract. The system may also provide the benefits of controlling the amount of bile available as signalling molecules for thyroid hormone receptors in order to control a patient's energy expenditure, as well as controlling the amount of bile available for glucagon-like peptide 1 (GLP-1) in order to control appetite, and manage Type II diabetes by controlling insulin release from the pancreas. Numerous methods and devices are disclosed herein for accomplishing these effects. These methods and devices fall under three general categories: bile diversion systems, bile manipulation systems, and surgical methods. The surgical methods disclosed fall under three general categories: bile diversion, bile exclusion, and bile acceleration.

Numerous bile diversion systems are disclosed herein. In general, these systems provide an inlet to some or all of the bile that exits the common bile duct, such that the bile is diverted to a more distal location in the body, such as in the gastrointestinal (GI) tract, to limit or prevent nutrient absorption in the body, and may optionally serve as a way for fixing a bile manipulation system within the GI tract. Three main bile diversion system types are disclosed. They include malabsorptive catheter systems, stents, and diversion valves, all of which will be discussed in greater detail later herein.

The bile manipulation systems disclosed herein generally serve as a means to prevent bile from interacting with food content in the GI tract. Two main bile manipulation system types are disclosed. They include thermal bile deactivating catheters and chemical bile deactivating catheters, both of which will be discussed in greater detail later herein.

Two main surgical methods for correcting an imbalance between caloric intake and caloric expenditure are disclosed herein. They include direct shunting of the common bile duct and connecting proximal and distal portions of the small intestine to speed delivery of bile and/or chyme to distal regions of the GI tract, both of which will be discussed in greater detail later herein. These surgical methods are minimally invasive when compared to known procedures and are fully reversible.

As may be appreciated, any of the aforementioned devices and methods disclosed herein may be practiced either alone or in combination with any of the other disclosed devices and methods, where practical, without departing from the scope of the present invention, to achieve the desired effect of correcting an imbalance between caloric intake and caloric expenditure to enable excess weight loss, which is non-invasive or minimally invasive, and may be reversible in nature.

### BRIEF DESCRIPTION OF THE DRAWINGS

The invention will be more fully understood from the following detailed description taken in conjunction with the accompanying drawings, in which:

FIG. 1 is a schematic partially transparent view of a clipped catheter system placed in the Ampulla of Vater.

FIG. 2 is a schematic partially transparent view of a self-expanding stent placed in the Ampulla of Vater.

FIG. 3 is a schematic partially transparent view of a self-expanding stent having a diversion valve placed in the Ampulla of Vater.

FIG. 4 is a schematic partially transparent view of a therapeutic system connected to the bile inlet of the duodenum through a transhepatic catheter.

FIG. 5 is a schematic partially transparent view of a therapeutic system connected to the bile inlet of the duodenum through a transhepatic catheter having a subcutaneous fill port.

FIG. 6 is a schematic partially transparent view of a therapeutic system and associated reservoir, pumping system and control system connected to the bile inlet of the duodenum through a transhepatic catheter having a subcutaneous fill port.

FIG. 7 is a schematic partially transparent view of a Y-connection between the bile inlet of the duodenum and a catheter.

FIG. 8 is a schematic partially transparent view of a catheter which extends into an impermeable length of tubing extending to the bile inlet of the duodenum.

FIG. 9 is a schematic partially transparent view of a catheter with a one-way check valve, which extends into an impermeable length of tubing extending to the bile inlet of the duodenum.

FIG. 10 is a schematic partially transparent view of a catheter which extends into an impermeable length of tubing extending into the bile inlet of the duodenum, and having inlet features therein.

FIG. 11 is a schematic partially transparent view of a bile pumping system to accelerate an intestinal braking effect in combination with a gastric band.

FIG. 12 is schematic partially transparent view of a catheter system for thermally deactivating a portion of the bile passing therethrough.

FIG. 13 is schematic partially transparent view of a forked catheter system for chemically deactivating a portion of the bile passing therethrough.

FIG. 14 is a schematic view of a direct diversion of the common bile duct by means of shunting and/or surgical bypass.

FIG. 15 is a schematic partially transparent view of an extraluminal catheter delivered distally in the small intestine by connecting proximal and distal portions of the small intestine.

FIG. 16 is a schematic partially transparent view of an extraluminal catheter delivered distally in the small intestine by connecting proximal and distal portions of the small intestine in an alternative manner.

FIG. 17 is a schematic partially transparent view of a stent and catheter system exiting the common bile duct at the Sphincter of Oddi, then travelling through the small intestine to the jejunum where it exits and travels subcutaneously along the abdominal wall into the systemic vein.

FIG. 18 is a schematic partially transparent view of a stent and dual catheter system exiting the common bile duct at the Sphincter of Oddi for diverting bile and pancreatic secretions separately.

FIG. 19 is a schematic partially transparent view of a stent and catheter system exiting the common bile duct through the cystic duct and gall bladder into the jejunum.

FIG. 20 is a schematic partially transparent view of a catheter anchored within the gall bladder by a coil, and passing through the common bile duct into the duodenum.

FIG. 21 is a schematic view detailing a procedure for cutting the common bile duct before it joins the duodenum and reattaching it at a distal location.

FIG. 22 is a schematic view detailing a procedure for cutting the tissue surrounding the Sphincter of Oddi and transplanting that cut tissue patch at a distal location.

FIG. 23 is schematic partially transparent view of a catheter anchored within the common bile duct for diverting bile to a distal location in the small intestine.

### DETAILED DESCRIPTION OF THE INVENTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Numerous bile diversion systems are disclosed herein. In general, these systems provide an inlet to some or all of the bile that exits the common bile duct, such that the bile is diverted to a more distal location in the body, such as in the GI tract, to limit or prevent nutrient absorption in the body, and may optionally serve as a way for fixing a bile manipulation system within the GI tract. Three main bile diversion system types are disclosed. They include catheter systems, stents, and diversion valves, all of which will be discussed in greater detail later herein.

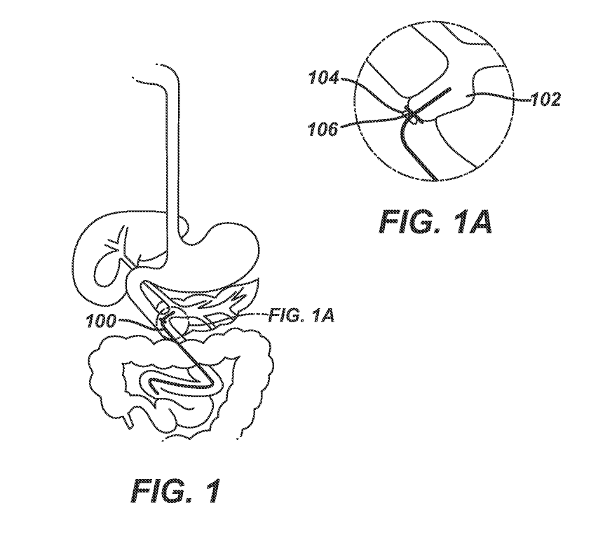

FIG. 1 is a schematic partially transparent view of clipped catheter system 100 placed in Ampulla of Vater 102 and secured therein at Sphincter of Oddi 104 using clip 106. In this embodiment, catheter 100 extends through the lumen of the duodenum and diverts biliary and pancreatic secretions approximately 125-175 cm distally from the Ligament of Treitz. Further, catheter 100 can be divided into two regions along its length, where each region is composed of a different absorbable material. The more proximal region of catheter 100 as best seen in the enlarged detail may be constructed from a material with a slower degradation rate than that of the distal region of catheter 100. Accordingly, the area of GI tract available for absorption of nutrients from food content would increase over time as catheter 100 degraded from its distal end to its proximal end. Examples of acceptable absorbable materials include polylactic acid/polylactide (PLA), polydimethylsiloxane (PDMS), polyglycolide/polyglycolic acid (PGA), and the like. Each of these materials absorbs in the body over time and has a different degradation rate generally ranging from several days to several months. Additionally, catheter 100 and/or clip 106 may further elute a therapeutic substance such as an antibiotic, a bile deactivating agent, an intestinal brake inducing agent, a hunger suppressing agent, or the like to further enhance the effectiveness of system 100.

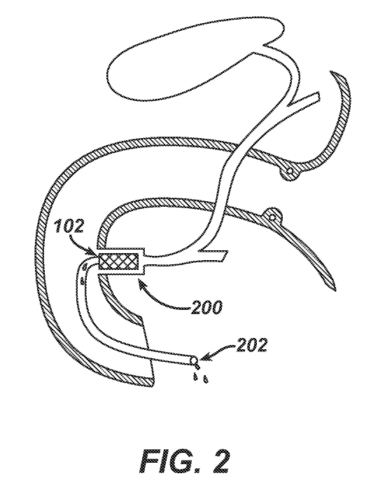

FIG. 2 is a schematic partially transparent view of self-expanding stent 200 placed in Ampulla of Vater 102. In this embodiment, stent 200 may be deployed endoscopically into Ampulla of Vater 102 by attaching it to a flexible, impermeable length of tubing 202 that extends down the intestinal lumen for a variable length. The purpose of tubing 202 is to shunt or divert biliary and pancreatic secretions away from the duodenum and proximal small intestine where they would normally mix with the contents thereof and be at least partially absorbed into the bloodstream. Due to the normal peristalsis of the GI tract, the biliary and pancreatic secretions within tubing 202 would be pumped therethrough. In certain embodiments, the tubing may be constructed from absorbable materials including polylactic acid/polylactide (PLA), polydimethylsiloxane (PDMS), polyglycolide/polyglycolic acid (PGA), and the like, and may further elute a therapeutic substance such as an antibiotic, a bile deactivating agent, an intestinal brake inducing agent, a hunger suppressing agent, or the like to further enhance the effectiveness of the system. Additionally, it may be desirous to anchor stent 200 within the Ampulla of Vater 102 via suturing, surface features on stent 200 or other known means.

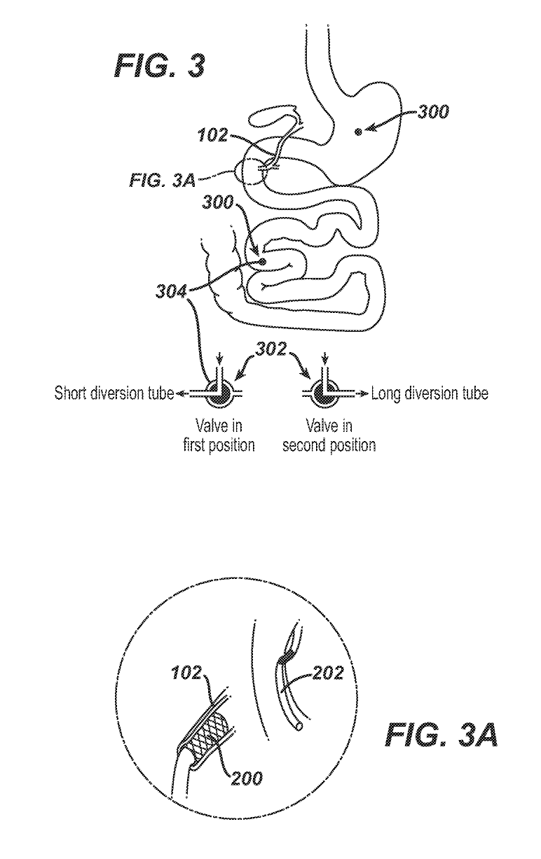

FIG. 3 is a schematic partially transparent view of self-expanding stent 200 having diversion valve 302 placed in Ampulla of Vater 102. In this embodiment, sensors 300 are provided at separate locations in the GI tract of a patient. Sensors 300 actively communicate with controller 304 to selectively control the position of valve 302. In this embodiment, valve 302 is a two position valve which is selectively controlled to permit flow of the biliary and pancreatic secretions to relatively proximal and relatively distal locations in the GI tract away from the duodenum and proximal small intestine where they would normally mix with the contents thereof and be at least partially absorbed into the bloodstream. In certain embodiments, controller 304 may comprise a portacath or transhepatic catheter through which a saline solution or the like may be injected or withdrawn to control the position of valve 302. In certain other embodiments, controller 304 may comprise an electronic controller.

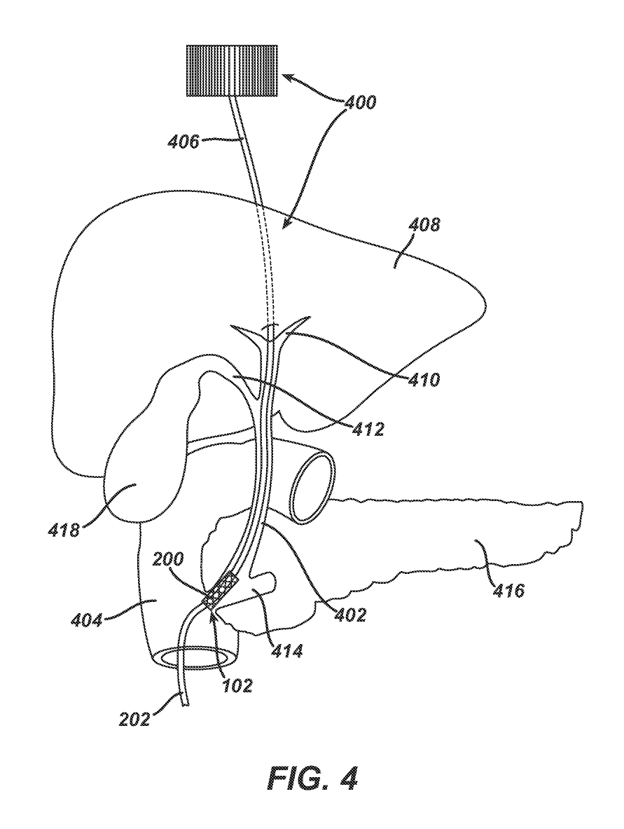

FIG. 4 is a schematic partially transparent view of therapeutic system 400 connected to the bile inlet of duodenum 404 through transhepatic catheter 406. In this embodiment, transhepatic catheter 406 passes through liver 408 and enters common bile duct 402 near hepatic ducts 410. Catheter 406 extends past gall bladder 418 and associated cystic duct 412, as well as pancreas 416 and associated pancreatic duct 414. As with previous embodiments, a self-expanding stent 200 is placed in Ampulla of Vater 102, and a flexible, impermeable length of tubing 202 extends down the intestinal lumen for a variable length. The purpose of tubing 202 is to shunt or divert biliary and/or pancreatic secretions away from the duodenum and proximal small intestine where they would normally mix with the contents thereof and be at least partially absorbed into the bloodstream. In this embodiment, a therapeutic substance such as an antibiotic, a bile deactivating agent, an intestinal brake inducing agent, a hunger suppressing agent, or the like may be supplied by catheter 406 to further enhance the effectiveness of system 400.

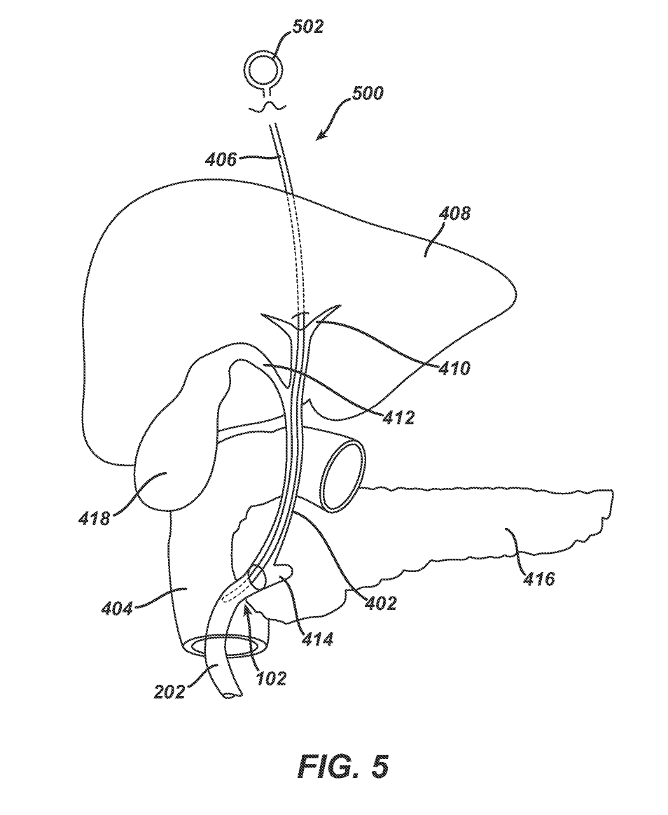

FIG. 5 is a schematic partially transparent view of therapeutic system 500 connected to the bile inlet of duodenum 404 through transhepatic catheter 406 having subcutaneous fill port 502. In the embodiment of FIG. 5, subcutaneous fill port 502 is provided as a way to provide a therapeutic substance to catheter 406. The therapeutic substance may, for example, comprise an antibiotic, a bile deactivating agent, an intestinal brake inducing agent, a hunger suppressing agent, or the like to further enhance the effectiveness of system 500. Additionally, tubing 202 is provided in a configuration that eliminates the need for stent 200; details of which are provided later herein with respect to FIG. 8.

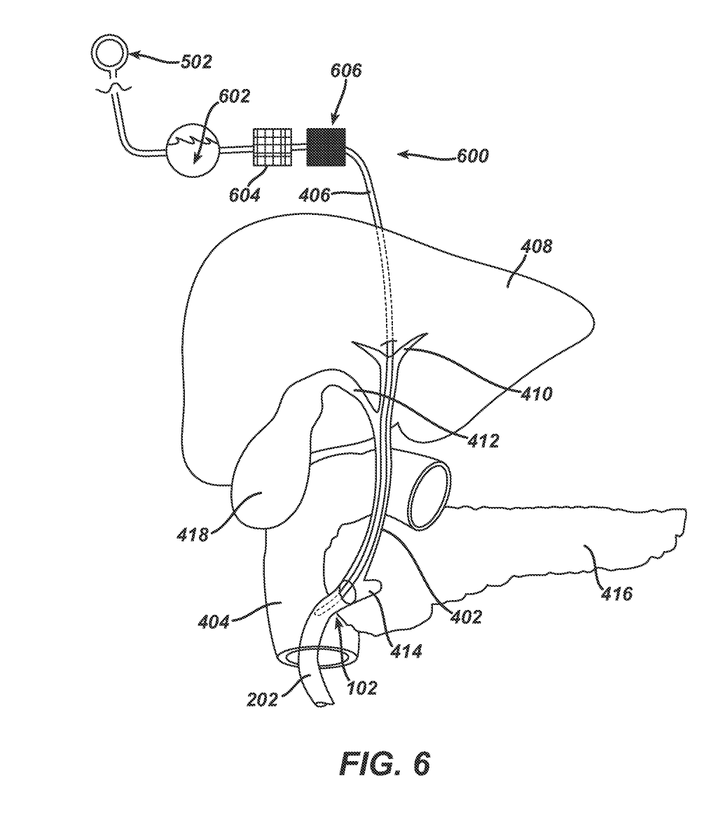

FIG. 6 is a schematic partially transparent view of therapeutic system 600 and associated reservoir 602, pumping system 604 and control system 606 connected to the bile inlet of duodenum 404 through a transhepatic catheter 406 having a subcutaneous fill port 502. In this embodiment, therapeutic substance is provided to reservoir 602 by subcutaneous fill port 502. The therapeutic substance within reservoir 602 is then pumped into catheter 406 via pumping system 604 as determined by control system 606. As with previous embodiments, the therapeutic substance may, for example, comprise an antibiotic, a bile deactivating agent, an intestinal brake inducing agent, a hunger suppressing agent, or the like to further enhance the effectiveness of system 600.

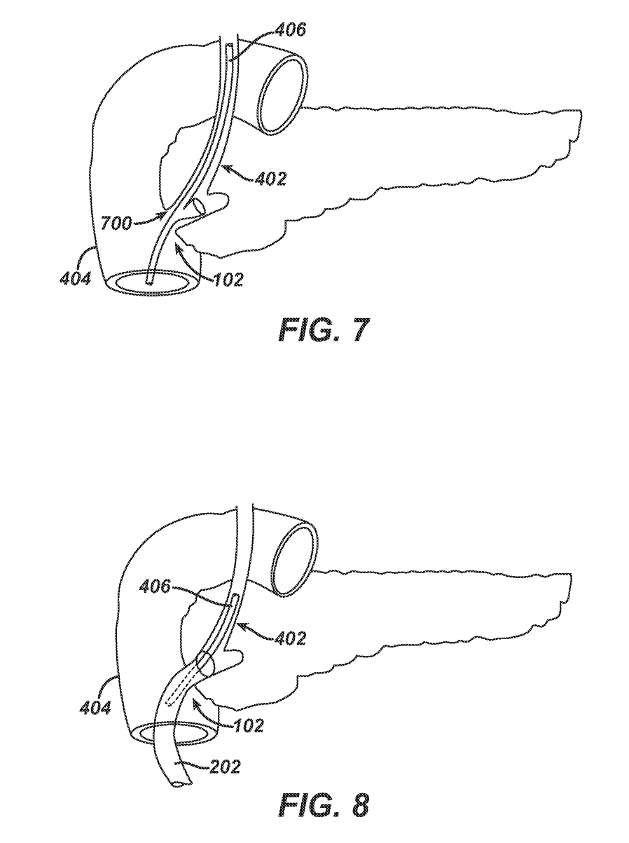

FIG. 7 is a schematic partially transparent view of Y-connection 700 between the bile inlet of duodenum 404 and transhepatic catheter 406. In this embodiment, there is no need for a stent to secure Y-connection 700 in Ampulla of Vater 102. Instead, Y-connection is supported by catheter 406, and is further sized and shaped to allow limited passage thereby of a portion of the biliary and pancreatic secretions in common bile duct 402. In this way, the risk of blockage of common bile duct 402 is greatly reduced, yet the advantages of delivery of the biliary and pancreatic secretions to relatively proximal and relatively distal locations in the GI tract away from the duodenum and proximal small intestine are realized.

FIG. 8 is a schematic partially transparent view of transhepatic catheter 406 which extends into an impermeable length of tubing 202 extending to the bile inlet of duodenum 404. The purpose of tubing 202 is to shunt or divert biliary and pancreatic secretions away from the duodenum and proximal small intestine where they would normally mix with the contents thereof and be at least partially absorbed into the bloodstream. In this embodiment, tubing 202 is provided in a configuration that eliminates the need for a separate stent to hold it in place. In this manner, the bile inlet of the duodenum 404 remains unaffected, whereas if a stent were used, it may be prone to tissue thickening as it grew to cover the stent. Further, in this configuration, the bile inlet of the duodenum 404 is maintained opened to prevent a build up of biliary and pancreatic secretions which may increase the risk of a blockage or infection, and further allows for a continuous shunting or diversion of biliary and pancreatic secretions away from the duodenum and proximal small intestine.

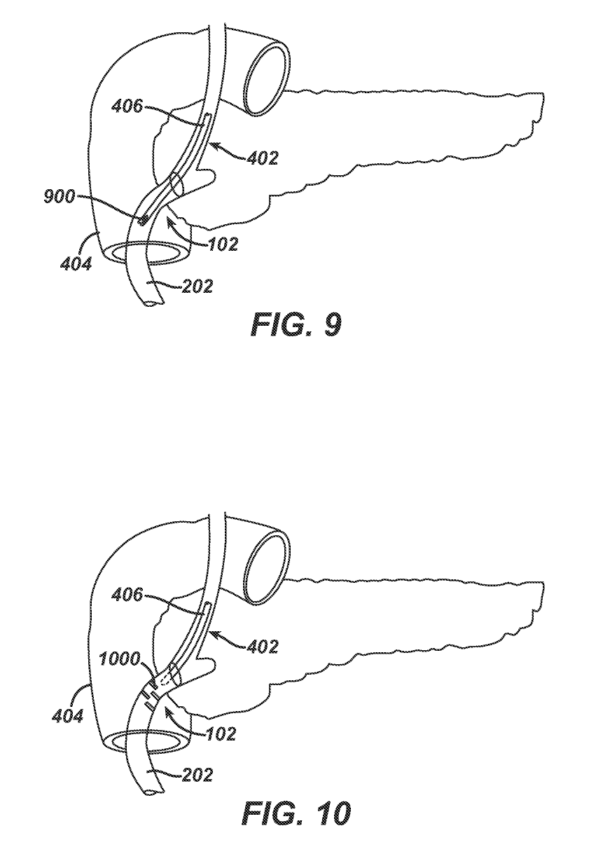

FIG. 9 is a schematic partially transparent view of transhepatic catheter 406 with a one-way check valve 900, which extends into an impermeable length of tubing 202 extending to the bile inlet of duodenum 404. As with previous embodiments, the purpose of tubing 202 is to shunt or divert biliary and pancreatic secretions away from the duodenum and proximal small intestine where they would normally mix with the contents thereof and be at least partially absorbed into the bloodstream. In this embodiment, check valve 900 is functional to prevent backflow from the GI tract into common bile duct 402, thereby reducing the risk of infection or blockage in common bile duct 402. Check valve 900 may be formed from very thin materials, such as a polyester having a thickness of less than one-thousandth of an inch, and as low as about two ten-thousandths of an inch or thinner. Thus, check valve 900 may be designed to accommodate flow of biliary and pancreatic secretions into the GI tract when the biliary and pancreatic secretions flow under very low pressure, but to still prevent backflow from the GI tract. Transhepatic catheter 406 may have walls that are thicker than the walls of check valve 900 to provide durability for catheter 406 while still designing check valve 900 to allow low pressure flow.

FIG. 10 is a schematic partially transparent view of transhepatic catheter 406 which extends into an impermeable length of tubing 202 extending into the bile inlet of duodenum 404, and having inlet features 1000 therein. In this embodiment, inlet features 1000 comprise a series of finger-like protrusions attached at their respective bases to the interior of tubing 202 and unattached at their distal ends, although numerous other configurations are envisioned. Inlet features 1000 are functional to prevent backflow from the GI tract into common bile duct 402, thereby reducing the risk of infection or blockage in common bile duct 402.

Although embodiments of transhepatic catheters are discussed, these embodiments could also be useful with a catheter that is not installed transhepatically. As a non-limiting example, a catheter that is not installed transhepatically could also provide a therapeutic substance, connect to fill port 502, support Y-connection 700, extend into a length of tubing 202, contain check valve 900, or contain inlet features 1000.

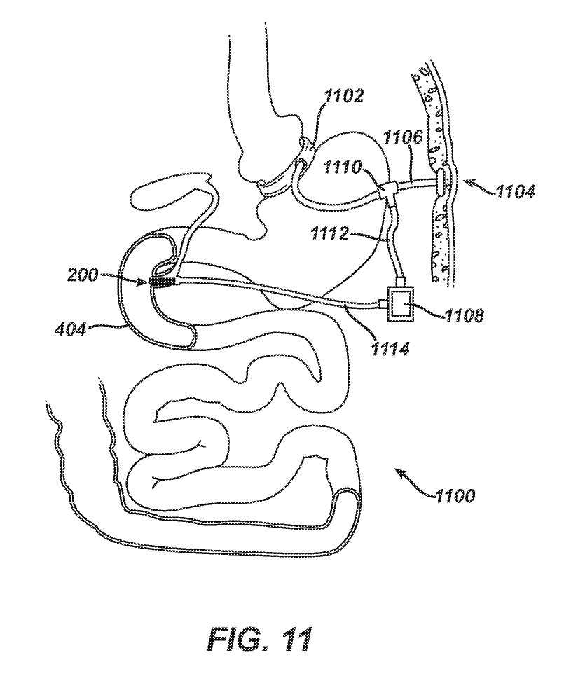

FIG. 11 is a schematic partially transparent view of bile pumping system 1100 to accelerate an intestinal braking effect in combination with gastric band 1102. In this embodiment, gastric band 1102 is connected to fill port 1104 via tubing 1106. This allows fluid to be added or withdrawn to change the interior diameter of gastric band 1102. Additionally, a mechanism 1108 is connected by tubing 1112 between gastric band 1102 and fill port 1104 via a T-fitting 1110 positioned along tubing 1106. Extending from mechanism 1108 is tube 1114 which is placed in Ampulla of Vater 102 by stent 200. Mechanism 1108 can serve many functions, such as being a fluid reservoir, a check valve system, a pre-programmed controller, and/or may be functional to move fluid in either or both directions along tubing 1112 and/or tubing 1114 separately or in concert. In certain other embodiments, gastric band 1102 may be operable to provide pressure signals to mechanism 1108 representative of the act of swallowing food, which would in turn enable mechanism 1108 to operate as a logic controller for the operation of the various components of system 1100. In this manner, mechanism 1108 can dynamically change the interior diameter of gastric band 1102 and/or provide fluid into the bile inlet of the duodenum 404 in order to cause flushing or pumping of biliary and pancreatic secretions more rapidly down the GI tract. The provided fluid may comprise, for example, a bile deactivating agent, an intestinal brake inducing agent, an antibiotic agent, a hunger suppressing agent, or the like to further enhance the effectiveness of the system. In alternative embodiments, a stent and extension tube system as in FIG. 2 may be used as a means to shunt or divert the provided fluid and biliary and pancreatic secretions away from the duodenum and proximal small intestine where they would normally mix with the contents thereof and be at least partially absorbed into the bloodstream. Thus, the provided fluid may be directed to the jejunum or the ileum, distally to the ampulla of Vater. Alternately, the provided fluid may be directed proximally to the ampulla of Vater.

Although not explicitly shown or discussed previously herein with respect to the bile diversion systems of FIGS. 1-11, it is anticipated that bile diversion to distal intestinal locations may limit the ability of bile to lower the pH level of chyme in proximal intestinal locations. Accordingly, the need may exist for pH balancing to accompany these bile diversion systems. Exemplary pH balancing devices include catheter systems formed of semi-permeable pH membranes, components which elute pH balancing substances, and the like. Such pH balancing devices would act to balance the pH levels of the chyme within the GI tract to the appropriate levels in order to prevent tissue damage within the GI tract due to overly acidic chyme.

As may be appreciated, numerous modifications to the systems disclosed in FIGS. 1-11 may be realized without departing from the scope of the present invention. For example, catheter 406 and/or tubing 202 may have indicia thereon to aid in determining their proper length during implantation. In other instances, catheter 406 may be installed surgically in common bile duct 402 and exit out through the lumen into the peritoneal space, then be reinserted into the desired distal GI tract location, thereby eliminating the need for peristalsis to pump the biliary and pancreatic secretions through tubing 202. Further, it should be understood that by rerouting the biliary and pancreatic secretions to distal locations in the GI tract, they are prevented from coming into contact with ingesta and chyme in the small intestine until they are far enough along in the GI tract that they cannot be or can only be minimally absorbed. It should also be understood that by shunting a small amount of undigested intestinal contents and/or biliary and pancreatic secretions to distal locations in the GI tract sooner than would be possible through natural biological processes, the patient may experience greater feelings of satiation, as well as prolonged feelings of satiation.

The bile manipulation systems disclosed herein generally serve as a way to prevent bile from interacting with food content in the GI tract, thereby preventing or limiting digestion and absorption of nutrients into the body. Two main bile manipulation system types are disclosed. They include thermal bile deactivating catheters and chemical bile deactivating catheters, both of which will be discussed in greater detail later herein.

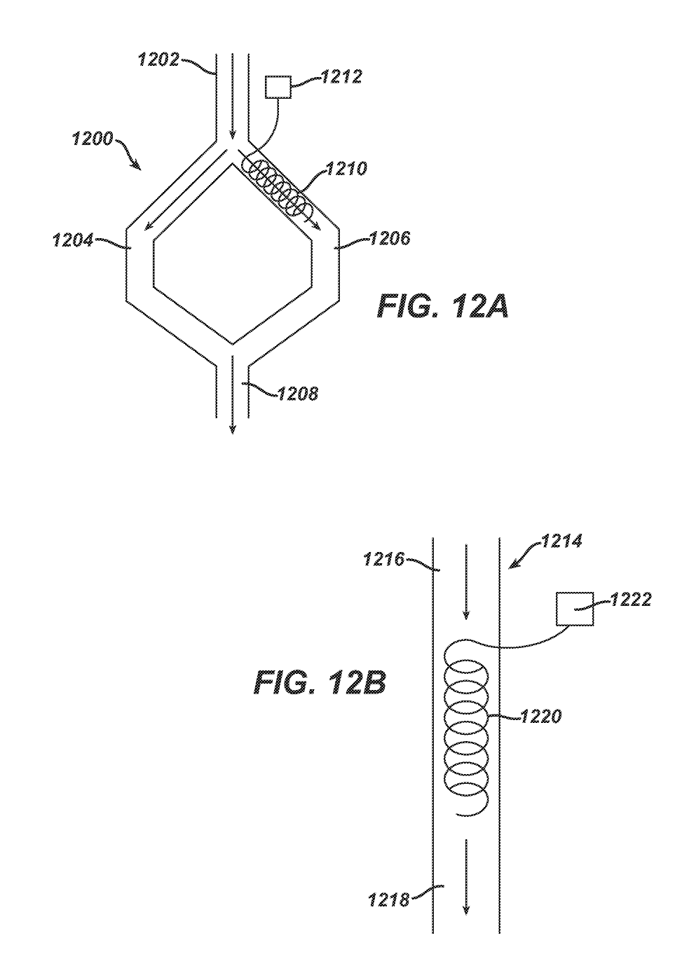

FIG. 12 is schematic partially transparent view of a catheter system for thermally deactivating a portion of the biliary and pancreatic secretions passing therethrough. In the embodiment of detail A of FIG. 12, forked catheter 1200 has an inlet lumen 1202 that is divided into a first branch 1204 and a second branch 1206 at its distal end which later reconnects at an outlet 1208 catheter. In this manner, secretions passing through catheter 1200 are divided between branch 1204 and branch 1206. Within branch 1206 resides a heating coil 1210 which is controlled by a controller 1212. In certain embodiments, controller 1212 may be a logic controller which uses sensed parameters to determine the operating characteristics of heating coil 1210. As secretions pass through branch 1206, heating coil 1210 (or other means for deactivating at least a portion of the digestive enzymes in an amount of secretions, as described below) is operable to impart thermal energy to the secretions to raise the temperature to a point where the digestive enzymes within the secretions are deactivated. The secretions passing through branch 1204 are left unaffected. In this manner, when the secretions of branches 1204 and 1206 are recombined at outlet 1208, the overall potency of the bile is decreased, thus decreasing its ability to aid in the digestion and absorption of nutrients in the body. In the embodiment of detail B of FIG. 12, catheter 1214 does not include the forked configuration of catheter 1200 of detail A. Instead, secretions enter catheter 1214 through inlet 1216 and passes over heating coil 1220 before exiting at outlet 1218. Heating coil 1220 is controlled by controller 1222 and may be operated such that it is pulsed on and off to create an alternating deactivated and unaffected secretions stream exiting from outlet 1218. In this manner, the average potency of the secretions exiting outlet 1218 is decreased, thus decreasing its ability to aid in the digestion and absorption of nutrients in the body.

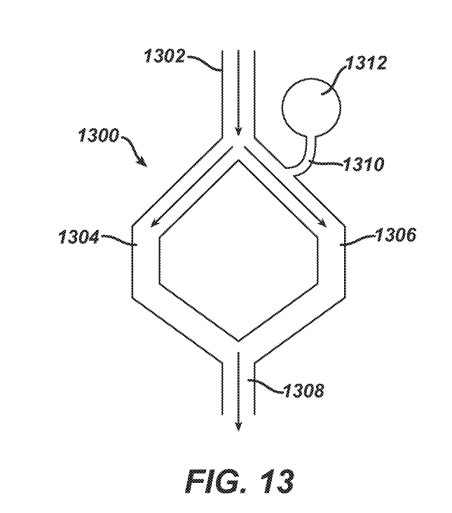

FIG. 13 is schematic partially transparent view of forked catheter system 1300 for chemically deactivating a portion of the biliary and pancreatic secretions passing therethrough. Forked catheter 1300 has an inlet 1302 that is divided into a first branch 1304 and a second branch 1306 which later reconnect at an outlet 1308. In this manner, secretions passing through catheter 1300 are divided between branch 1304 and branch 1306. Connected to branch 1306 via tube 1310 is reservoir 1312. In certain embodiments, a logic controller which uses sensed parameters to determine the operating characteristics of a pump connected to reservoir 1312 may be employed to selectively supply a biliary and pancreatic secretion neutralizing agent to the secretion stream flowing through branch 1306. In this manner, as secretions pass through branch 1306, the supplied secretion neutralizing agent deactivates the digestive enzymes within the secretions. The secretions passing through branch 1304 are left unaffected. In this manner, when the secretions of branches 1304 and 1306 are recombined at outlet 1308, the overall potency of the secretions is decreased, thus decreasing its ability to aid in the digestion and absorption of nutrients in the body. In certain embodiments, a valve means may be included in tube 1310 which may also be controlled by the logic controller outlined above. Further, certain embodiments may also include a therapeutic eluting stent. As with previous embodiments, the therapeutic substance being eluted may comprise, for example, a pH balancing fluid, a biliary and pancreatic secretion deactivating agent, an intestinal brake inducing agent, an antibiotic agent, a hunger suppressing agent, or the like to further enhance the effectiveness of the system.

Two main surgical methods for correcting an imbalance between caloric intake and caloric expenditure are disclosed herein. They include direct shunting of the common bile duct and connecting proximal and distal portions of the small intestine to speed delivery of bile and/or chyme to distal regions of the GI tract, both of which will be discussed in greater detail later herein. These surgical methods are minimally invasive when compared to known procedures and are fully reversible.

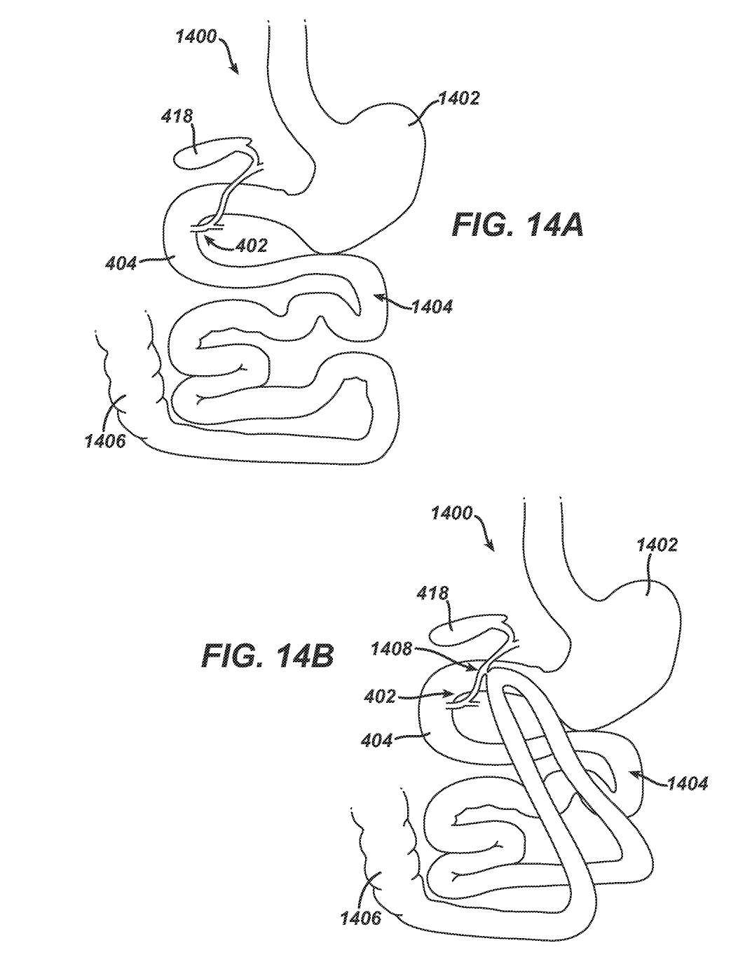

FIG. 14 is a schematic view of a direct diversion of common bile duct 402 by means of shunting and/or surgical bypass. Detail A of FIG. 14 illustrates an unaffected GI tract having an esophagus 1400, stomach 1402, duodenum 404, small intestine 1404, large intestine 1406, gall bladder 418, and common bile duct 402. In detail B of FIG. 14, a distal portion of small intestine 1404 is brought into fluid communication with common bile duct 402 at location 1408, via shunting and/or surgical bypass. This may be accomplished, for example, by a side-to-side anastomosis of common bile duct 402 and small intestine 1404. In this manner, at least a portion of the biliary and pancreatic secretions within common bile duct 402 are channelled away from duodenum 404 and proximal portions of small intestine 1404 where they would normally mix with the contents thereof and be at least partially absorbed into the bloodstream. In certain embodiments, a direct diversion of the Papilla of Vater by means of shunting or surgical bypass external to the GI tract may be employed as a means to allow bile, chyme and the like to flow directly to the ileum which would creating an intestinal braking effect.

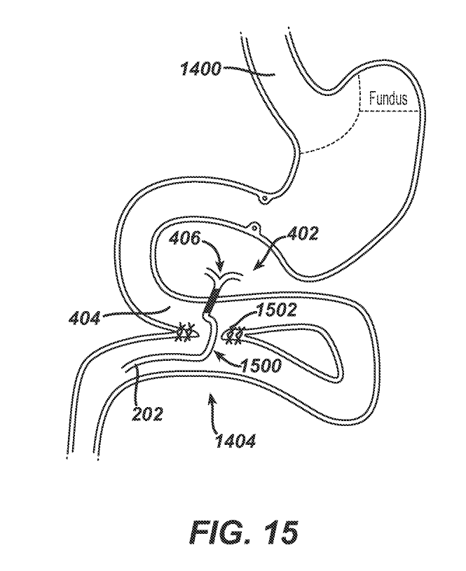

FIG. 15 is a schematic partially transparent view of extraluminal catheter 406 delivered distally in small intestine 1404 by connecting proximal and distal portions of small intestine 1404. In one embodiment, the proximal portion of small intestine 1404 may comprise the duodenum 404 while the distal portion of small intestine 1404 may comprise a middle portion of the ileum. In this embodiment, the proximal and distal portions of small intestine 1404 may be stapled or sutured together by fasteners 1502, leaving an opening 1500 which is large enough for tubing 202 and a small amount of chyme to pass therethrough. In this manner, the biliary and pancreatic secretions within common bile duct 402 are channelled away from duodenum 404 and proximal portions of small intestine 1404 where they would normally mix with the contents thereof and be at least partially absorbed into the bloodstream, and the chyme passing through opening 1500 may aid is speeding the initiation of an intestinal brake in the GI tract. This further allows for the biliary and pancreatic secretions within common bile duct 402 to be directly channelled to the ileum which would create an intestinal braking effect. As with previous embodiments of the present invention, it is anticipated that bile diversion may limit the ability of bile to lower the pH level of chyme in proximal intestinal locations. Accordingly, the need may exist for pH balancing to accompany such systems. Examples of such pH balancing devices include catheter systems formed of semi-permeable pH membranes or components which elute pH balancing substances. Such pH balancing would act to balance the pH levels of the chyme within the GI tract to the appropriate levels in order to prevent tissue damage within the GI tract due to overly acidic chyme.

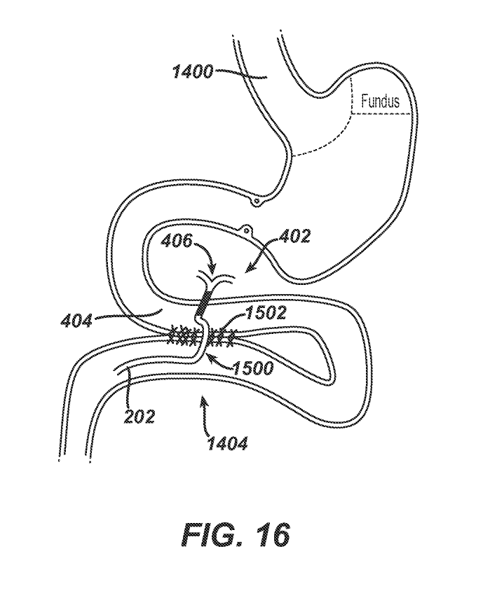

FIG. 16 is a schematic partially transparent view of an extraluminal catheter 406 delivered distally in small intestine 1404 by connecting proximal and distal portions of small intestine 1404 in an alternative manner. In this embodiment, the proximal and distal portions of small intestine 1404 may be stapled or sutured together by fasteners 1502, leaving an opening 1500 which is large enough for only tubing 202 to pass therethrough. In this manner, the biliary and pancreatic secretions within common bile duct 402 are channelled away from duodenum 404 and proximal portions of small intestine 1404 where they would normally mix with the contents thereof and be at least partially absorbed into the bloodstream, while chyme must still pass through the length of small intestine 1404. As with previous embodiments of the present invention, it is anticipated that bile diversion may limit the ability of bile to lower the pH level of chyme in proximal intestinal locations. Accordingly, the need may exist for pH balancing means to accompany such systems. Examples of such pH balancing means include catheter systems formed of semi-permeable pH membranes or components which elute pH balancing substances. Such pH balancing means would act to balance the pH levels of the chyme within the GI tract to the appropriate levels in order to prevent tissue damage within the GI tract due to overly acidic chyme.

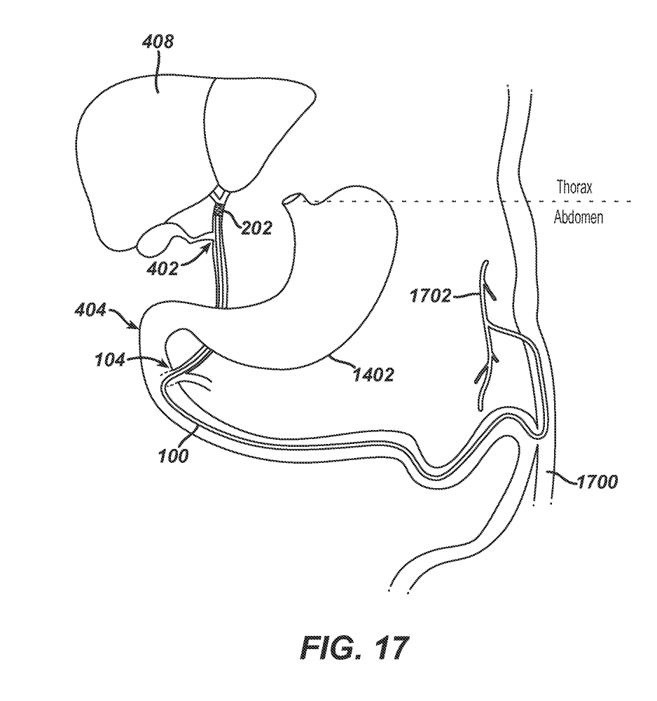

FIG. 17 is a schematic partially transparent view of stent 200 and catheter 100 system exiting common bile duct 402 at Sphincter of Oddi 104, then travelling through the small intestine to the jejunum where it exits and travels subcutaneously along abdominal wall 1700 into the systemic vein 1702. Alternately, catheter 100 system could travel into the thoracic duct. The purpose of this procedure is to divert at least a portion of the bile from common bile duct 402 to systemic vein 1702 or the thoracic duct. This procedure may be accomplished via a conventional endoscopic retrograde cholangiopancreatography (ERCP). It should be noted that the jejunum may be affixed to abdominal wall 1700, in order to prevent leakage where catheter 100 exits the jejunum and enters abdominal wall 1700. Additionally, as with other embodiments disclosed herein, a pumping system (not shown) or valve (not shown) may be further included to compensate for pressure differentials and to prevent backflow through catheter 100 into common bile duct 402.

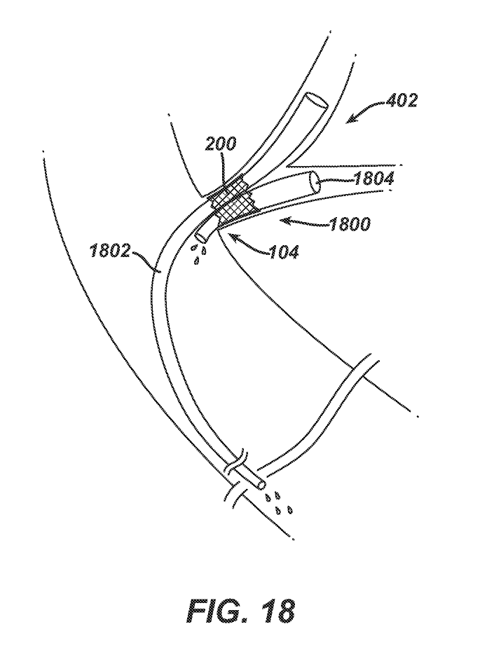

FIG. 18 is a schematic partially transparent view of stent 200 and dual catheter system 1800 exiting common bile duct 402 into the small intestine at Sphincter of Oddi 104, for diverting bile and pancreatic secretions separately. In one embodiment, first catheter 1802 is positioned such that it diverts only bile, while second catheter 1804 is positioned such that it diverts only pancreatic secretions. In the embodiment shown in FIG. 18, first catheter 1802 is longer than second catheter 1804, however, it should be understood that any desirable lengths may be chosen for these catheters at the discretion of the attending physician, so as to enable the desired metabolic improvement in the patient. As may be appreciated, catheter 1804 may be absent in certain embodiments of the invention, leaving the pancreatic secretions to flow normally without diversion.

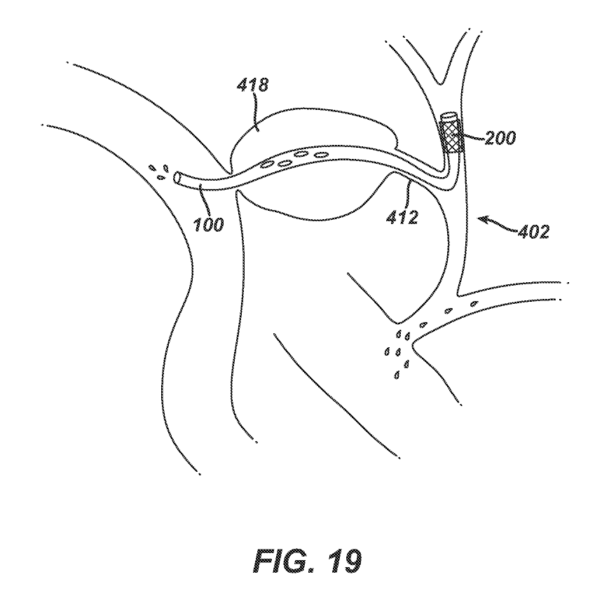

FIG. 19 is a schematic partially transparent view of stent 200 and catheter 100 exiting common bile duct 402 through cystic duct 412 and gall bladder 418 into the jejunum. In one embodiment, an anastomosis of gall bladder 418 and jejunum may be performed. In another embodiment, gall bladder 418 and jejunum may simply be fenestrated to allow passage of catheter 100 therethrough. In certain embodiments, catheter 100 may also be fixated at its distal end and/or at the cholecysto jejunostomy location in order to maintain its position and allow bile to freely flow therethrough. As may be appreciated, in addition to the jejunum location disclosed in FIG. 18, various other locations in the body may be realized for the diverted bile as have been disclosed herein with respect to other embodiments, without departing from the scope of the present invention. Further, catheter 100 can be fenestrated in its location within gall bladder 418 in order to allow the free flow of bile to and from gall bladder 418, in case catheter 100 were to completely obstruct cystic duct 412.

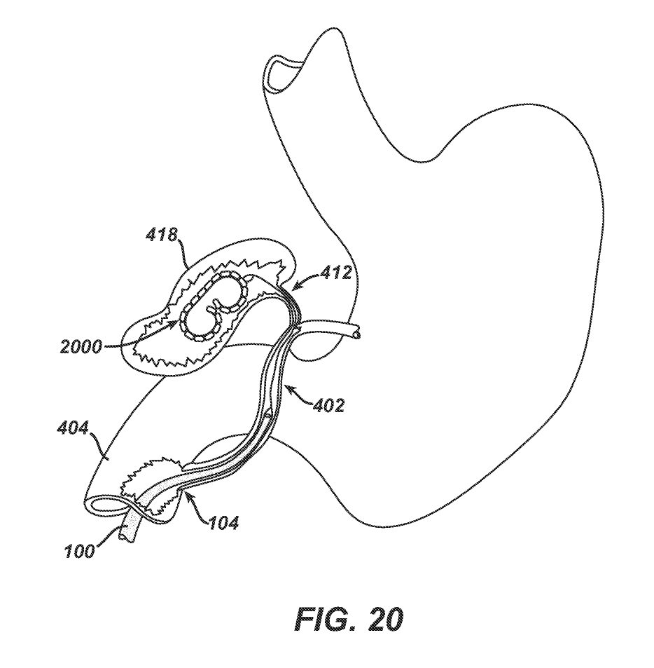

FIG. 20 is a schematic partially transparent view of catheter 100 anchored within gall bladder 418 by coil 2000, and passing through common bile duct 402 into duodenum 404. Coil 2000 may, for example, be implanted by passing it through duodenum 404, past Sphincter of Oddi 104, through common bile duct 402, into cystic duct 412, arriving in gall bladder 418, where it may be expanded and deployed to serve as an anchor for catheter 100.

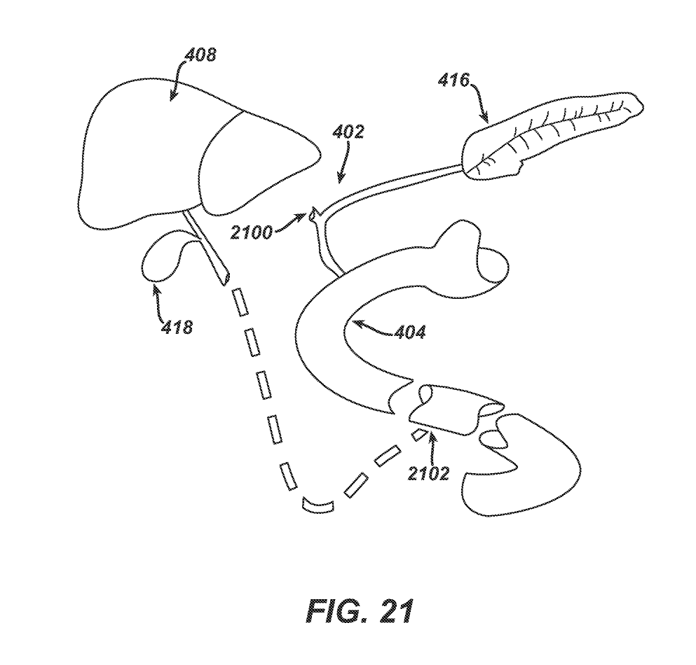

FIG. 21 is a schematic view detailing a procedure for cutting common bile duct 402 at location 2100, before it joins duodenum 404, and reattaching it at a distal location 2102. In this particular embodiment, distal location 2102 is the ileum, however, as with the other embodiments disclosed herein, alternate locations are contemplated. Additionally, as with other embodiments disclosed herein, a pumping system (not shown) or valve (not shown) may be further included to compensate for pressure differentials and to prevent backflow through common bile duct 402.

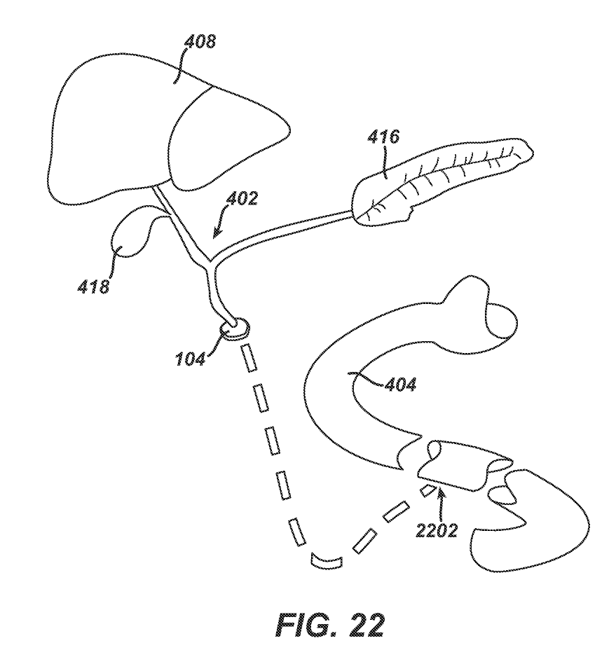

FIG. 22 is a schematic view detailing a procedure for cutting the tissue surrounding Sphincter of Oddi 104 and transplanting that patch of tissue at distal location 2202. In this particular embodiment, distal location 2202 is the ileum, however, as with the other embodiments disclosed herein, alternate locations are contemplated. Additionally, as with other embodiments discloses herein, a pumping system (not shown) or valve (not shown) may be further included to compensate for pressure differentials and to prevent backflow through common bile duct 402.

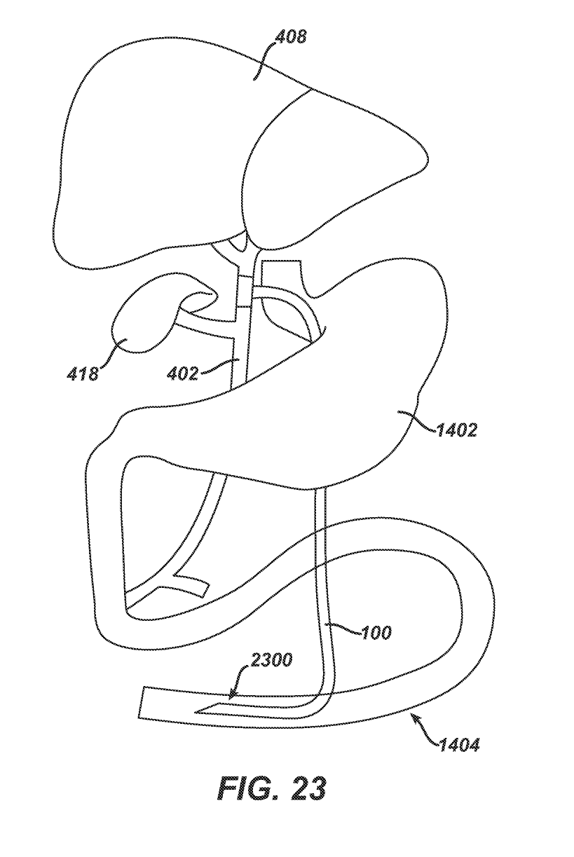

FIG. 23 is schematic partially transparent view of catheter 100 anchored within common bile duct 402 for diverting bile to a distal location 2300 in small intestine 1404. In this particular embodiment, distal location 2300 is the ileum, however, as with the other embodiments disclosed herein, alternate locations are contemplated. Additionally, as with other embodiments discloses herein, a pumping system (not shown) or valve (not shown) may be further included to compensate for pressure differentials and to prevent backflow through catheter 100 into common bile duct 402.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A catheter for surgically treating a patient comprising:
a. an inlet catheter having a proximal end and an open distal end;
b. first and second branch sections having proximal ends attached to said open distal end of said inlet lumen and distal ends terminating at an open proximal end of an outlet lumen;
c. said first passageway lumen having a means for deactivating at least a portion of the digestive enzymes in an amount of bile.

2. The catheter of claim 1, wherein said means for deactivating at least a portion of the digestive enzymes in an amount of bile comprises least one of: thermal and chemical means.
